(19) United States
(12) Patent Application Publication
(10) Pub. No.: US 2026/0348958 A1
(43) Pub. Date: Nov. 5, 2026

(54) METHODS USING ST2 TO IDENTIFY PATIENTS FOR CANCER THERAPY

(71) Applicant: BOARD OF REGENTS, THE UNIVERSITY OF TEXAS SYSTEM, Austin, TX (US)

(72) Inventors: Swetha ANANDHAN, Houston, TX (US); Padmanee SHARMA, Houston, TX (US); Sangeeta GOSWAMI, Houston, TX (US)

(73) Assignee: BOARD OF REGENTS, THE UNIVERSITY OF TEXAS SYSTEM, Austin, TX (US)

(21) Appl. No.: 19/487,971

(22) PCT Filed: May 24, 2024
(86) PCT No.: PCT/US2024/031094

§ 371 Date: Nov. 25, 2025

Related U.S. Application Data
(60) Provisional application No. 63/504,400, filed on May 25, 2023.

(51) Int. Cl. G01N 33/569; C12Q 1/6869

(52) U.S. Cl. CPC G01N 33/56972; C12Q 1/6869; C12Q 2600/158; G01N 2333/7155; G01N 2800/52

(57) ABSTRACT

Aspects herein relate to methods, compositions, and systems directed to the discovery that ST2+ Tregs in a tumor, in some instances, leads to a poor response to immune checkpoint therapies in the tumor. Such findings provide methods and compositions for diagnosing, prognosing, and treating certain cancers.

### BACKGROUND OF THE INVENTION

This application claims priority of U.S. Provisional Patent Application No. 63/504,400, filed May 25, 2023, which is hereby incorporated by reference in its entirety.

### I. FIELD OF THE INVENTION

This invention relates to the field of oncology, pathology, and medicine.

### II. BACKGROUND

The use of immune checkpoint therapy (ICT) in cancer treatment has significantly improved clinical outcomes; however, only 20-30% of patients experience long-lasting durable effects. Combination therapies that target different pathways/cells have not achieved great success due to the lack of predictive biomarkers for selecting patients who may benefit from ICT.

### SUMMARY OF THE INVENTION

In general, the current invention relates to the discovery that ST2+ Tregs in a tumor, in some instances, leads to a poor response to immune checkpoint therapies in the tumor. Such findings provide methods and compositions for diagnosing, prognosing, and treating certain cancers.

Certain aspects related to methods of detecting ST2 in a biological sample comprising Tregs. Certain aspects relate to methods of assaying ST2 in a population of cells. Certain aspects relate to methods of identifying ST2 in a population of cells. The disclosed methods can comprise 1, 2, 3, 4, 5, or more of any of the following steps: processing a biological sample including to obtain a population of cells, detecting ST2 in a biological sample or the population of cells, comparing detected levels of ST2 to a reference level, determining a therapeutic intervention based on the detected levels of ST2, and administering a therapeutic intervention. In certain aspects, the population of cells is a population of lymphocytes, T-cells, or Tregs. In some aspects, the population of cells are from an individual that has, or is suspected of having cancer. In some aspects, the method comprises the step of detecting ST2 in in a biological sample. The biological sample may comprise or may not comprise Tregs from an individual. The individual may have, or is suspected of having, a cancer. The cancer may be a candidate for an immune checkpoint therapy. Cancers that are candidates for an immune checkpoint therapy are known in the art, such as any cancer described herein. In some aspects, the biological sample comprising Tregs comprises a tumor biopsy. The tumor biopsy may be a solid tumor biopsy. The tumor biopsy may be collected in any manner described herein.

In some aspects, the biological sample is enriched for lymphocytes. In certain aspects, the biological sample is enriched for Tregs. In certain aspects, specific lymphocytes, such as B cells, cytotoxic T cells, and/or dendritic cells, are not enriched. The enrichment may be by centrifugation, affinity purification, cell sorting, or any other method known in the art.

In certain aspects, the biological sample is prepared for immunohistochemistry. The biological sample may be fixed in a particular medium suitable for immunohistochemistry. The biological sample may be formalin fixed and sectioned. In certain aspects, detecting ST2 comprises detecting by immunohistochemistry. In some aspects, the detecting by immunohistochemistry comprises contacting the biological sample, which may be fixed and/or stained, with an antibody and/or reagent capable of staining the ST2 present in the biological sample. In certain aspects, detecting ST2 comprises detecting by sequencing. The sequencing may be any sequencing suitable for determining the expression profile of a cell. In certain aspects, the sequencing comprises RNA sequencing. The RNA sequencing may comprise single cell RNA sequencing. In certain aspects, the immune checkpoint therapy comprises an anti-PD1 composition and/or an anti-CTLA4 composition, or other agent capable of inhibiting or suppressing the immune checkpoint machinery. In certain aspects, the immune checkpoint therapy does not comprise an anti-PD1 composition. In certain aspects, the immune checkpoint therapy does not comprise an anti-CTLA4 composition.

In some aspects, detecting ST2 comprising detecting ST2 protein or other ST2 gene products. Detecting ST2 may be by any method, including immunohistochemistry. In some aspects, detecting ST2 protein comprises detecting by Western blot, ELISA, cytometry, chromatography, mass spectrometry, and/or immunoprecipitation.

In certain aspects, detecting ST2 comprises detecting ST2 mRNA. Detecting ST2 mRNA may comprise any method of detecting specific mRNA, including by polymerase chain reaction (PCR). The PCR may be a reverse transcriptase PCR (RT-PCR). The PCR may be quantitative PCR (qPCR). The PCR may be quantitative RT-PCR (RT-qPCR). In certain aspects, detecting TSG-6 mRNA comprises sequencing. The sequencing may be any sequencing suitable for determining the expression profile of a cell. In certain aspects, the sequencing comprises RNA sequencing. The RNA sequencing may comprise single cell RNA sequencing.

Certain aspects relate to methods of treating a cancer in an individual. Certain aspects relate to method of reducing the severity of a cancer in an individual. Certain aspects relate to methods of preventing symptoms associated with cancer in an individual. In some aspects, the method comprises the step of administering a non-immune checkpoint therapy to an individual who has been determined to have ST2+ Tregs in an amount substantially equal to or greater than a reference level. The non-immune checkpoint therapy may comprise a chemotherapy, immunotherapy, biologic, cell therapy, radiation, surgery, or the like. In certain aspects, the reference level is any amount, including a detectable amount, of ST2+ Tregs, which may be present in the individual. In certain aspects, the reference level is determined by measuring ST2+ Tregs in two or more biological samples from the individual. The biological samples may have been taken before, during, or after a therapy. The biological sample may have been taken prior to the individual being diagnosed with cancer. In certain aspects, the reference level comprises an amount of ST2+ Tregs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

In some aspects, the method comprises the step of administering an immune checkpoint therapy to an individual who has been determined to have ST2+ Tregs in an amount less than a reference level. The biological samples may have been taken before, during, or after a therapy. The biological sample may have been taken prior to the individual being diagnosed with cancer. In certain aspects, the reference level is a detectable amount of ST2+ Tregs present in the individual. In certain aspects, the reference level is determined by measuring ST2+ Tregs in two or more biological samples from the individual. In certain aspects, the reference level comprises an amount of ST2+ Tregs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

Certain aspects relate to methods of prognosing the effectiveness of an immune checkpoint therapy in an individual, the method comprising the step of measuring the presence of ST2+ Tregs to get a measurement and comparing the measurement to a reference. In certain aspects, the immune checkpoint therapy comprises an anti-PD1 composition and/or an anti-CTLA4 composition. In certain aspects, the reference is a detectable amount of ST2+ Tregs present in the individual. In certain aspects, the reference is determined from measuring ST2+Tregs in two or more biological samples from the individual. In certain aspects, the reference comprises an amount of ST2+ Tregs found in an individual responsive to an immune checkpoint therapy or a healthy individual. The individual may be considered responsive to the immune checkpoint therapy, or any other therapy described herein, when the therapy causes the individual to have a reduction in symptoms, reduction in tumor burden, reduction in tumor aggressiveness, reduction in tumor size, reduction in metastasis, and/or improvement in quality of life. A healthy individual may be an individual that does not have cancer or does not have cancer that is refractory to an immune checkpoint therapy.

Certain aspects relate to methods of treating a cancer in an individual. In some aspects, the method comprises the step of administering a composition comprising a therapeutically effective amount of an anti-ST2 agent. In certain aspects, the anti-ST2 composition comprises an antibody targeting ST2, an RNA interference composition targeting ST2, and/or a small molecule capable of inhibiting ST2. In certain aspects, the method further comprises administering a therapeutically effective amount of an immune checkpoint therapy. In certain aspects, the composition comprises the immune checkpoint therapy. In certain aspects, the composition does not comprise the immune checkpoint therapy. In certain aspects, the composition and immune checkpoint therapy are given concurrently. Concurrently may be in the same composition or in separate compositions. Concurrently may be that the anti-ST2 agent is given within 1, 2, 3, 4, 5, 6, 7, 8, 9, 10 or more, and any range derivable therein, minutes, hours, or days of the immune checkpoint therapy. In certain aspects, the composition and immune checkpoint therapy are given sequentially. In certain aspects, the immune checkpoint therapy comprises an anti-PD1 composition and/or an anti-CTLA4 composition. In certain aspects, the cancer is refractory to and immune checkpoint therapy. In certain aspects, the cancer is a myeloid-rich cancer. In certain aspects, the cancer is a pancreatic cancer (which may be a pancreatic ductal adenocarcinoma), a glioblastoma, and/or a prostate cancer. In certain aspects, the cancer is a melanoma.

Certain aspects relate to methods for determining a treatment for an individual with cancer, the method comprising the step of administering a non-checkpoint inhibitor therapy to an individual found to have an amount of ST2+ Tregs equal to or greater than a reference. In certain aspects, the detectable amount of ST2+ Tregs is found by measuring the expression of ST2 in at least one biological sample from the individual. In certain aspects, the biological sample comprises a tumor biopsy. In certain aspects, the biological sample is enriched for lymphocytes. In certain aspects, the biological sample is enriched for T-cells. In certain aspects, the biological sample is enriched for Tregs. In certain aspects, the biological sample is prepared for immunohistochemistry. In certain aspects, the reference is a detectable amount of ST2+ Tregs present in the individual. In certain aspects, the reference is determined from measuring ST2+ Tregs in two or more biological samples from the individual. In certain aspects, the reference comprises an amount of ST2+ Tregs found in an individual responsive to an immune checkpoint therapy or a healthy individual.

The following definitions may be used in describing the invention and claims.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1F: (A) (left) UMAP plot of intratumoral T cells in B16F10 and mT4 tumors at baseline. Cell clusters have been defined based on the marker gene expression levels. (right) UMAP plots of T cells split by tumor type highlighting the presence of distinct Tregs in both tumors (red circle). (B) Heatmap of gene expression of top genes in all cell clusters identified in the scRNAseq dataset. (C) Plot of cluster frequency ratios of each immune subset in B16F10 and mT4 tumors. (D) Heatmap of functional markers for the indicated T cell subsets providing phenotypic information. Expression levels are scaled between minimum and maximum expression for each gene across all clusters. (E) Differential gene expression plot in the two Treg subsets. (F) Expression of indicated genes in the two Treg subsets.

FIGS. 2A-2C: (A) UMAP plot of intratumoral T cells in B16F10 and mT4 tumors post ICT treatment (B) UMAP plots for each treatment group in both the tumor models. ISG+Tregs (B16F10_Treg) are highlighted in green dotted circles and ST2+ Tregs (MT4_Treg) are highlighted in red dotted circles. (C) Cluster frequency of Tregs isolated from B16F10 and mT4 tumors upon ICT treatment.

FIGS. 3A-3D: (A) Schematic of experimental set up to perform longitudinal immune analysis. (B) UMAP plot of cell distribution. (C) Cluster frequency of Tregs sorted from mT4 tumors on indicated days using scRNAseq. (D) Heatmap indicating change in gene expression in the Treg subset with time.

FIGS. 4A-4E: (A) Quantification of multi-immunofluorescence (mIF) images after IL-33 staining human pancreatic and melanoma FFPE sample. (B) (left) Representative mIF image indicating presence of CD4+FoxP3+ST2+ cells in human pancreatic tissue (white arrow), (right) quantification of the images. (C) Representative UMAP plot of baseline ICT responders and non-responders melanoma patients indicating presence of two Treg clusters (Cluster 1 and 10) (D) Heatmap of gene expression markers in C1 and C10 Treg cluster. (E) GSEA plot indicated upregulated pathways in C1 compared to C10 Treg subsets.

FIGS. 5A-5B: (A) Survival analysis of the B16F10 tumor bearing mice randomized into the four treatment groups (n=10). (B) Survival analysis of the mT4 tumor bearing mice randomized into the four treatment groups. (n=10).

FIGS. 6A-6C: (A) Bar plot indicated total cells assessed using scRNAseq analysis post ICT from all groups. (B) Cluster frequency of indicated CD8 T cell subsets post ICT treatment in both tumor models. (C) Cluster frequency of indicated CD4 T cell subsets post ICT treatment in both tumor models.

### DETAILED DESCRIPTION OF THE INVENTION

Certain aspects herein concern single cell RNA sequencing (scRNAseq), including on melanoma and pancreatic tumors and murine models thereof that are sensitive (responsive) and resistant (non-responsive) to immune checkpoint therapy (ICT), respectively. Also disclosed are distinct subsets of regulatory T cells (Tregs) present in tumors. In certain aspects, the ICT-sensitive melanoma tumors contain Tregs expressing interferon stimulated genes (ISG+ Tregs), whereas the ICT-resistant pancreatic tumors contain Tregs expressing suppressive genes such as Klrg1, Areg and Il1rl1, which encodes for the IL-33 receptor, also called ST2 (ST2+ Tregs). In certain aspects, the ISG+ Tregs are depleted from the tumors upon combination therapy of anti-CTLA-4 and anti-PD-1, while the ST2+ Tregs are not affected by either single or combination ICT treatment. Also disclosed are analyses of a published scRNAseq dataset of patients with metastatic melanoma. In some aspects, baseline non-responders have ST2+ Tregs, indicating presence of ST2+ Tregs as markers of ICT non-responsive tumors. Also disclosed are ST2 markers used as biomarkers to identify patients who will benefit from ICT combined with agents targeting Tregs. In some aspects, this can be detected through IHC assays.

### I. Therapeutic Methods

Methods of the disclosure relate to treating subjects and patients with a cancer therapy. The cancer therapy may be one described herein, including an immune checkpoint therapy and/or an agent that targets ST2, and may be given with respect to a patient having been determined to have a certain biomarker profile. The agent that targets ST2 may be an anti-ST2 antibody, an RNA interference composition that targets ST2, or an inhibitor of ST2. The RNA interference composition may be an siRNA, miRNA, or other noncoding RNA. The biomarker profile may be the presence or absence of one or more biomarkers expressed on or by a Treg cell. The biomarker may comprise all or part of ST2. In some aspects, the therapy described below is given to a patient with a poor prognosis, unfavorable prognosis, or to a patient determined to be high risk. In some aspects, the therapy is given to a patient having a prognosis that reflects a favorable outcome for an immune checkpoint therapy. In some aspects, the therapy described below is given to a patient with a favorable prognosis, or to a patient determined to be low risk. In some aspects, the therapy is given to a patient having a prognosis that reflects an unfavorable outcome for an immune checkpoint therapy. Also contemplated are combinations of the therapies described below.

The compositions of the disclosure, including agents that target ST2, may be used for in vivo, in vitro, or ex vivo administration. The route of administration of the composition may be, for example, intratumoral, intracutaneous, subcutaneous, intravenous, local, topical, and intraperitoneal administrations.

### A. Immune Checkpoint Therapies and Combination Treatment

Aspects of the disclosure may include administration of immune checkpoint therapies, also known as checkpoint inhibitors, which are further described below. The administration of the immune checkpoint therapies may be done in combination with one or more other therapies, such as an agent that targets ST2. In some aspects, pharmacological drugs, biologics, nucleic acids, and/or blocking antibodies are generated against ST2 which in combination with an immune checkpoint therapy will improve clinical response in patients, including those with myeloid rich tumors like pancreatic tumors, glioblastoma and prostate cancer. In some aspects, ST2 is used to develop immunohistochemistry assays that will enable selection patients who should receive, and/or may be responsive to, anti-ST2 optionally with an immune checkpoint therapy.

### 1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### B. Administration of Therapeutic Compositions

The therapy provided herein may comprise administration of a combination of therapeutic agents, including a first therapy, such as an immune checkpoint therapy, and a second therapy, such as an agent that targets ST2. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

In some aspects, the first therapy and the second therapy are administered substantially simultaneously. In some aspects, the first therapy and the second therapy are administered sequentially. In some aspects, the first therapy, the second therapy, and a third therapy are administered sequentially. In some aspects, the first therapy is administered before administering the second therapy. In some aspects, the first therapy is administered after administering the second therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, the first therapy is administered at a dose of between 1 mg/kg and 5000 mg/kg, or any range derivable therein. In some aspects, the first therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

In some aspects, a single dose of the second therapy is administered. In some aspects, multiple doses of the second therapy are administered. In some aspects, the second therapy is administered at a dose of between 1 mg/kg and 5000 mg/kg, or any range derivable therein. In some aspects, the second therapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range derivable therein.

The quantity to be administered, both according to number of treatments and unit dose, depends on the treatment effect desired. An effective dose is understood to refer to an amount necessary to achieve a particular effect. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM. ; or about 1 μM to 100 μM; or about 1 μM to 50 μM; or about 1 μM to 40 μM; or about 1 μM to 30 μM; or about 1 μM to 20 μM; or about 1 μM to 10 M; or about 10 μM to 150 μM; or about 10 μM to 100 μM; or about 10 μM to 50 MM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### C. Cancer Therapy

In some aspects, the method further comprises administering a cancer therapy to the patient. The cancer therapy may be chosen based on the expression level measurements, alone or in combination with the clinical risk score calculated for the patient. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is recurrent cancer. In some aspects, the cancer is Stage I cancer. In some aspects, the cancer is Stage II cancer. In some aspects, the cancer is Stage III cancer. In some aspects, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### D. Pharmaceutical Compositions

In certain aspects, the compositions or agents for use in the methods, such as an immune checkpoint therapy and/or an agent that targets ST2, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as a tumor or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono-or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal, tumoral, or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

In some aspects, pharmaceutical compositions are administered to a subject. Different aspects may involve administering an effective amount of a composition to a subject. In some aspects, an antibody or antigen binding fragment capable of binding to [protein of interest] may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions, such as any antibody described herein, may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. Diagnostic and Prognostic Methods

Certain aspects relate to diagnosing or prognosing an individual. The methods may be used to determine the effectiveness of one or more therapies in treating cancer in the individual. The therapies may comprise an immune checkpoint therapy and/or an agent that targets ST2.

### A. Sample Preparation

In certain aspects, methods involve obtaining a sample from a subject. The subject may have, or be suspected of having cancer. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

In certain aspects, the sample is prepared for

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### B. Protein Assays

A variety of techniques can be employed to measure expression levels of polypeptides and proteins, including ST2, in a biological sample to determine biomarker expression levels. Examples of such formats include, but are not limited to, enzyme immunoassay (EIA), radioimmunoassay (RIA), Western blot analysis, immunohistochemistry, and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, . beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### C. Monitoring

In certain aspects, the biomarker-based method may be combined with one or more other cancer diagnosis or screening tests at increased frequency if the patient is determined to be at high risk for recurrence or have a poor prognosis based on the biomarker as described above.

In some aspects, the methods of the disclosure further include one or more monitoring tests. The monitoring protocol may include any methods known in the art. In particular,
the monitoring include obtaining a sample and testing the sample for diagnosis. For example, the monitoring may include endoscopy, biopsy, laparoscopy, colonoscopy, blood test, genetic testing, endoscopic ultrasound, X-ray, barium enema x-ray, chest x-ray, barium swallow, a CT scan, a MRI, a PET scan, or HER2 testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### III. Kits

Certain aspects of the present disclosure also concern kits containing compositions of the disclosure or compositions to implement methods disclosed herein. In some aspects, kits can be used to evaluate one or more biomarkers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some aspects, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1x, 2x, 5x, 10x, or 20x or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

Any aspect of the disclosure involving specific biomarker by name is contemplated also to cover aspects involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Aspects of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

### A. Detection Kits and Systems

One can recognize that based on the methods described herein, detection reagents, kits, and/or systems can be utilized to detect the biomarkers, including ST2, for diagnosing or prognosing an individual. The reagents can be combined into at least one of the established formats for kits and/or systems as known in the art. The kits could also contain other reagents, chemicals, buffers, enzymes, packages, containers, electronic hardware components, etc. The kits/systems could also contain packaged sets of PCR primers, oligonucleotides, arrays, beads, antibodies, or other detection reagents. Any number of probes could be implemented for a detection array. In some aspects, the detection reagents and/or the kits/systems are paired with chemiluminescent or fluorescent detection reagents. Particular aspects of kits/systems include the use of electronic hardware components, such as DNA chips or arrays, or microfluidic systems, for example. In specific aspects, the kit also comprises one or more therapeutic or prophylactic interventions in the event the individual is determined to be in need of.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1

### ST2+ Tregs and Immune Checkpoint Therapies

Upon comparative analyses on the tumor infiltrating lymphocytes between murine tumor models sensitive to immune checkpoint therapy (ICT) (melanoma) and resistant to ICT (pancreatic tumors), the inventors identified the specific presence of IL-33 receptor (ST2) regulatory T cells (Tregs) in the pancreatic tumors as compared to interferon-stimulating genes (ISG) expressing Tregs in melanoma tumors. The ST2+ Tregs do not deplete from the pancreatic tumors post monotherapy or combination of anti-CTLA-4 and anti-PD-1 whereas the Tregs in melanoma tumor decrease with monotherapy and are eliminated post combination therapy. Further, the ST2+ Tregs increases exponentially as PDAC tumors progress. Presence of ST2+ Tregs was also observed at baseline in melanoma patients who do not respond to immunotherapy, indicating their role in ICT.

### Example 2

### Baseline MT4 Tumors Have Abundant TH2 Subsets and Low Cytotoxic CD8T Cells Whereas B16F10 Tumors Have More Effector and Functional T Cells in the TME

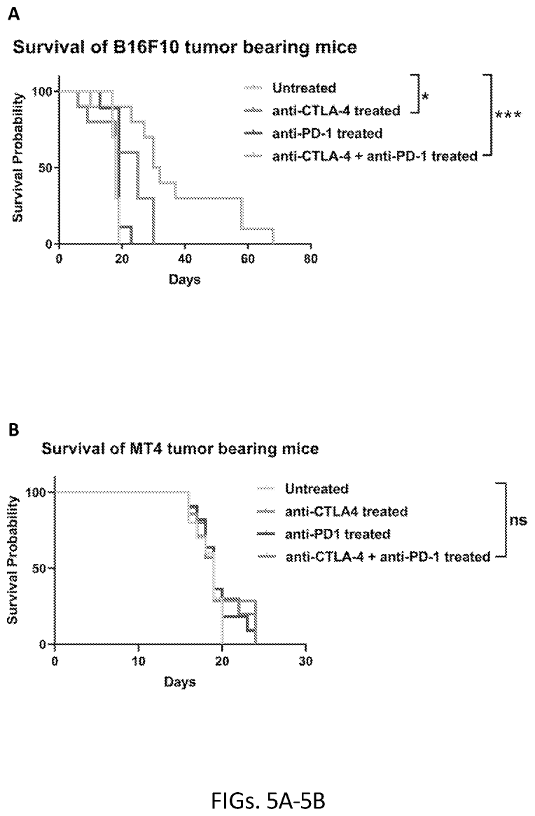

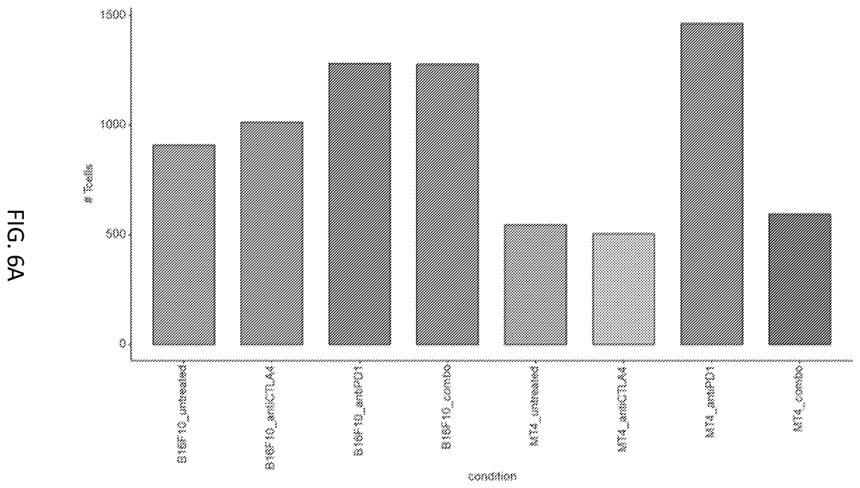

To investigate underlying mechanisms associated with immune checkpoint therapy (ICT) responses, the inventors utilized B16F10 melanoma and mT4-2D pancreatic ductal adenocarcinoma (PDAC) as the ICT-sensitive (responsive) and resistant (non-responsive) tumor models for this study. Mice bearing B16F10 tumors show significantly improved survival benefit when treated with anti-CTLA-4 and anti-PD-1 checkpoint antibodies (FIG. 5A). In contrast, mice bearing mT4 tumors show no survival benefit with either monotherapy or combination ICT therapy as compared to the control untreated mice (FIG. 5A) making these ideal tumor models for the study.

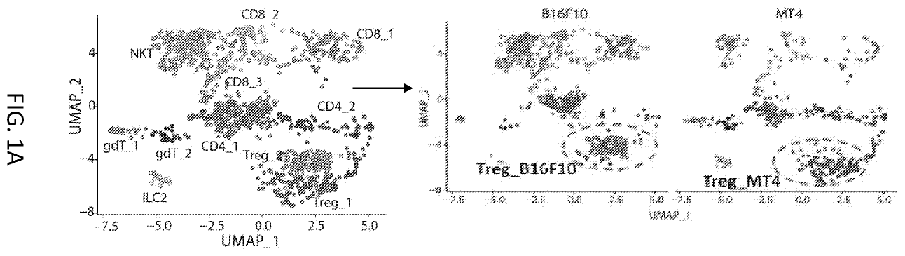

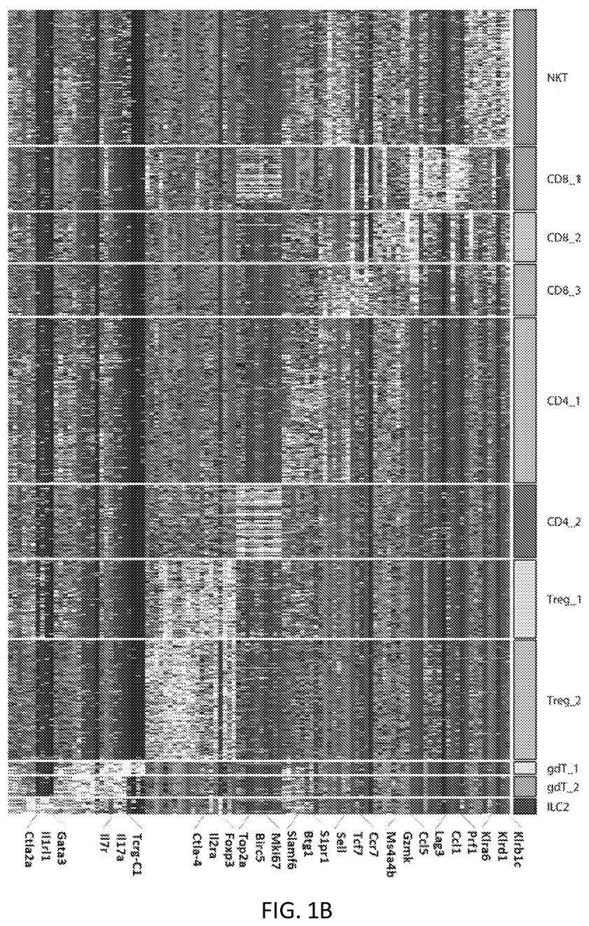

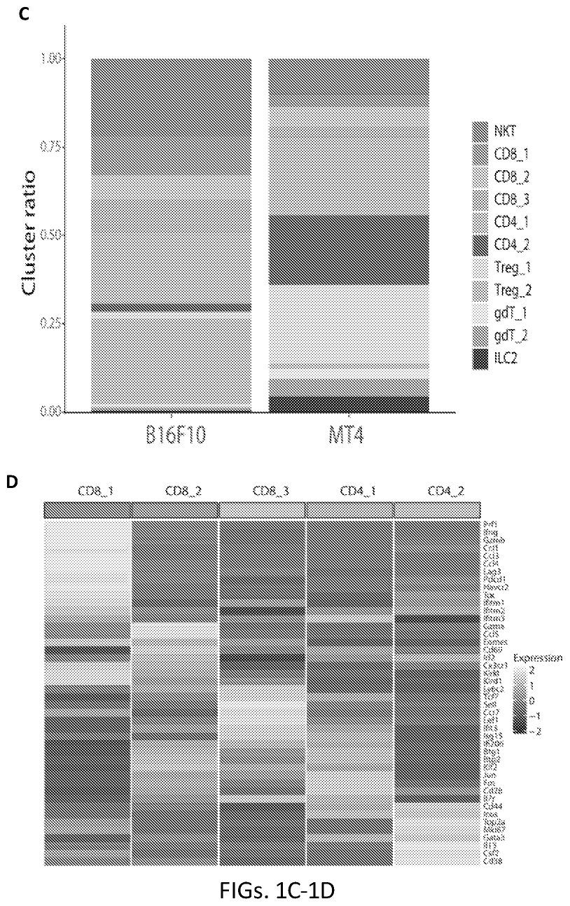

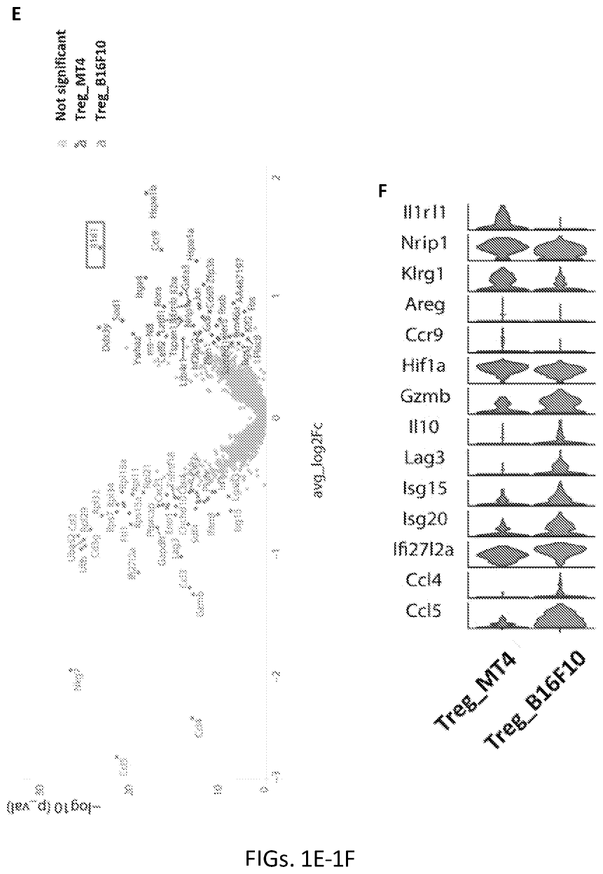

Next, to understand the differences in the tumor infiltrating immune cells in these models, the inventors isolated CD45 positive cells from untreated B16F10 and mT4 tumors and conducted single cell RNA sequencing (scRNAseq). The analysis identified a total of 11 subsets consisting of three CD8+T cell subsets, four CD4+T cell subsets, including two regulatory T cell (Tregs) clusters, two gdT subsets, and one subset each of innate lymphoid cells 2 (ILC2) and NKT cells (FIG. 1A-C).

The abundance of total CD8T cells was higher in B16F10 tumors when compared to mT4 tumors (FIG. 1A, C). Among the CD8T cells, CD8_1 was the most abundant cluster in B16F10 tumors and expressed cytotoxic genes such as Gzmb, Prf1, and Ifng, as well as inhibitory molecules like Lag3, Pdcd1 (encodes PD-1), Hvacr2 (encodes TIM-3), and Tox, a transcriptional factor known to regulate exhaustion (PMID: 31207603) (FIG. 1D). These characteristics indicate a terminally exhausted phenotype (hereafter referred to as CD8Texhausted). CD8_2, which was present in both tumor models, expressed activation markers (Eomes, Cd69) and effector genes (Gzma, Klrc, Klrd, Id2) but did not express any Prf1, Gzmb, or exhaustion and inhibitory markers, indicating an activated effector phenotype that was not as functional as CD8_1 (referred to as CD8Tactivated) (FIG. 1D). CD8_3 subset highly expressed Tcf7, Sell (encodes for CD62L), Lef1, Ccr7, along with some interferon stimulated genes (Ifit3, Isg15, Ifi206), indicating a progenitor central memory-like phenotype (referred to as CD8Tcm) (PMID:18209024). This subset was more prevalent in B16F10 tumors.

The inventors observed two primary non-Treg CD4+ T cell subsets in the two tumor models. CD4_1 was present in both models, while CD4_2 was more prevalent in mT4 tumors. CD4_1 subset expressed genes associated with cell stress (Jun, Fos), Gata3, interferon-induced genes (Ifitm3, Ifit3, Isg15, Ifi206), and traits that signify an effector memory phenotype (Cd44+Sell−Ccr7−Cd28+Il7r+) (hereafter referred to as CD4Tem). On the other hand, CD4_2 was specific to mT4 tumors and displayed Th2-like characteristics with Gata3, Il13, proliferation, and cell cycle genes (Ki67, Top2a), indicating a proliferative Th2-like CD4 T cell population (FIG. 1D).

In summary, the inventors found that B16F10 tumors exhibited a higher abundance of effector and progenitor-like CD8T and CD4T cells, while mT4 tumors were dominated by less functional CD8T cells and Th2 T cells. These observations indicate a distinct T cell microenvironment between the two tumor models.

### Example 3

### ICT-Sensitive and Resistant Tumors Have Distinct Treg Subsets Present in their TME

The inventors identified two distinct Treg clusters in both tumor models, with one cluster present exclusively in mT4 and the other in B16F10 (FIG. 1A-C). Further examination of the phenotypes revealed that Tregs in mT4 tumors expressed Il1rl1 (encodes for IL33 Receptor/stimulation 2 (ST2)), Nrp1, Klrg1, Areg1, Hif1a, and Ccr9 (referred to as ST2+Tregs), while Tregs in B16F10 tumors expressed higher levels of Il10, Gzmb, Ccl4, Ccl5, Lag3, and interferon-stimulating genes Isg15, Isg20, and Ifi2712a (referred to as ISG+Tregs) (FIG. 1E-F). This highlights the presence of distinct Treg subsets in the TME, which is often overlooked when using Foxp3 expression as a marker to identify Tregs in flow cytometry. Differential gene expression analysis revealed that Il1rl1 was the most enriched gene in mT4 Tregs (FIG. 1E), which is known to play a role in Treg-mediated suppressive functions (PMID: 31801068, 32376804).

### Example 4

### ICT Treatment Decreased ISG+ Tregs and Increased Effector T Cell Abundance in B16F10 Tumors but did not Alter the ST2+Treg Abundance in the MT4 Tumors

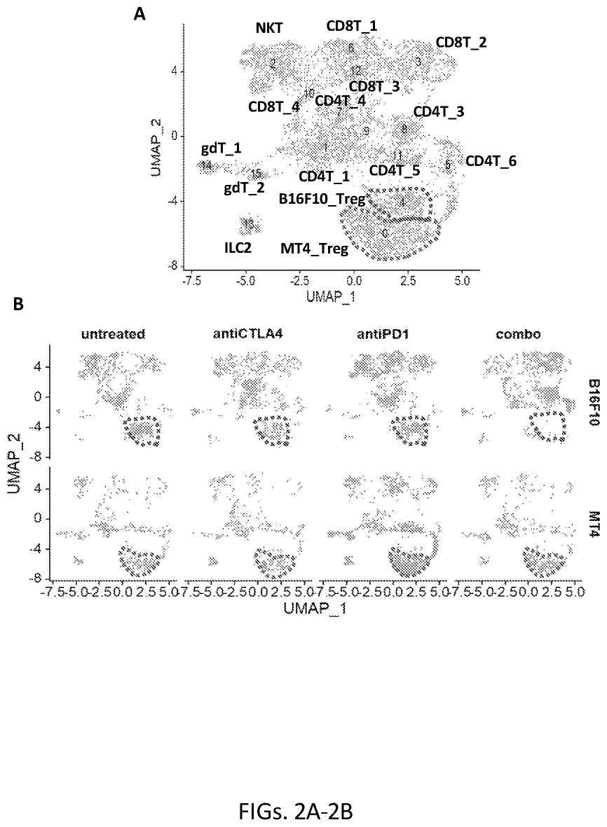

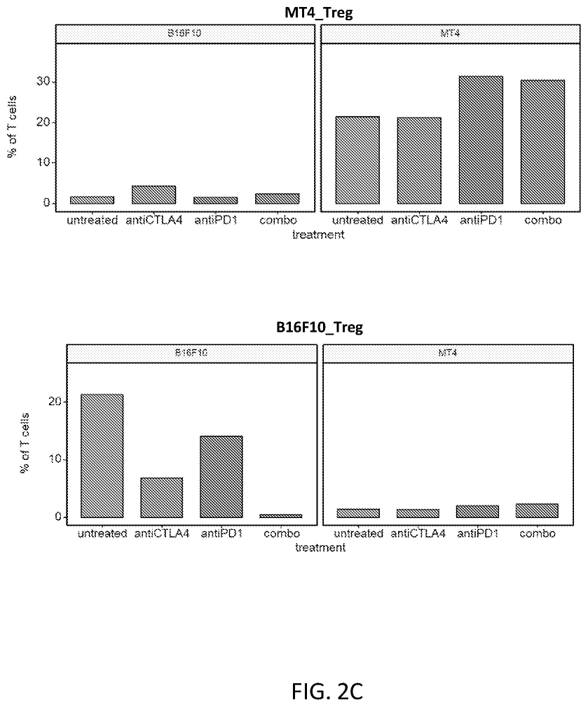

Following the characterization of the T cell microenvironment at baseline, the next objective was to investigate any changes in these immune subsets following ICT. The inventors conducted another scRNAseq analysis on Day 10 tumors, which included additional treatment groups receiving monotherapy or a combination of anti-CTLA-4 and anti-PD-1 in both tumor models (FIGS. 2A-B, 6A). The number of clusters increased from 11 to 15 after therapy, which included a newly identified CD8T cell subset, gdT cells, and four subsets of CD4T cells.

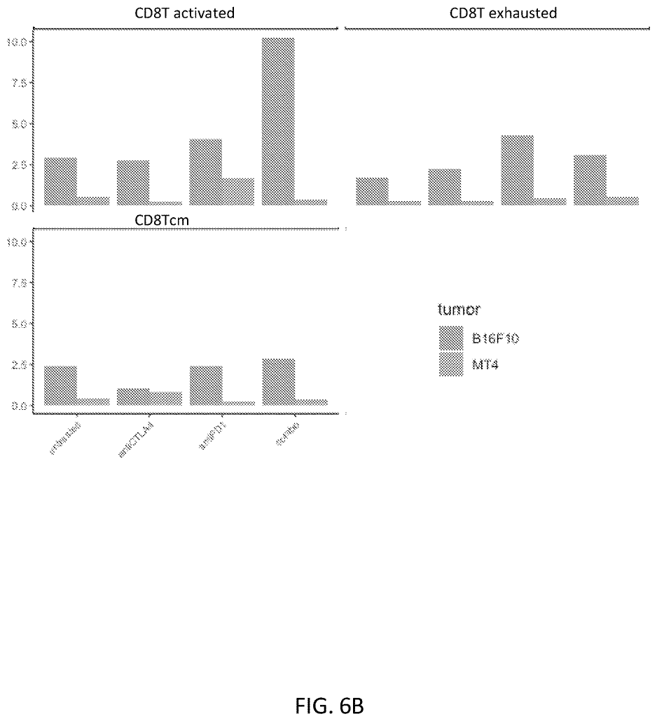

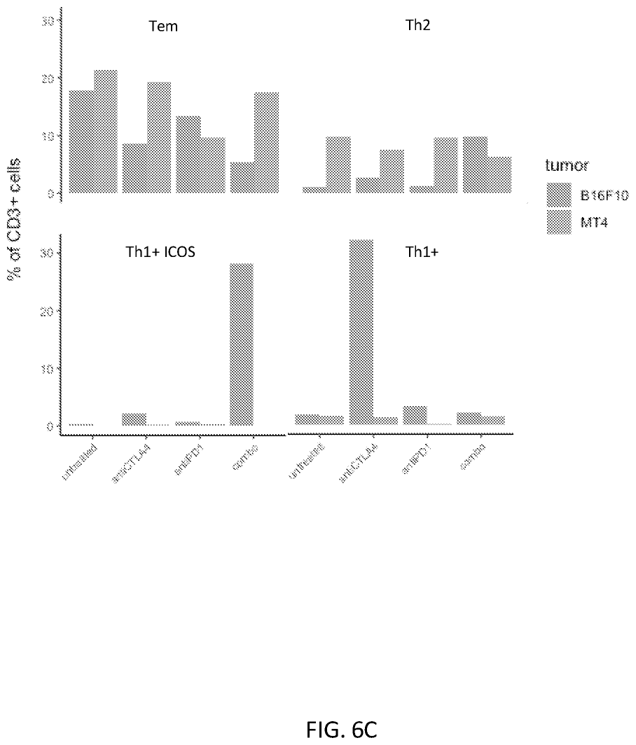

Although there was not much change in CD8Tcm and CD8T exhausted subsets, the inventors observed a increase in CD8T activated cells following ICT, the prevalence of CD8T exhausted subset increased drastically post ICT (FIG. 6B), which is indicative of ICT response and effectiveness, consistent with previous findings in B16F10 tumors (as described in PMID: 30778252 and 31606264). No dramatic changes were observed in CD8T cells in the mT4 tumors (FIG. 6B). These results suggest that while ICT revitalizes CD8T effector cells in B16F10 tumors, it does not increase either infiltration or effector-like phenotype in pancreatic tumors.

In the CD4 compartment, there were notable changes post-therapy. CD4Tem and Th2 subsets in mT4 tumors remained unchanged, but in B16F10 tumors, CD4Tem subset decreased post-therapy, while two new CD4 subsets emerged with anti-CTLA-4 therapy alone or in combination (FIG. 2C). These cells expressed Tbx21, activation markers (Icos, Cd69, Id2) and interferon-stimulated genes (Ifi206, Ifi209, Isg15), resembling activated Th1 and Th1 ICOS+ effector cells. Such cells have been shown to increase upon intervention with anti-CTLA-4 therapy in other ICT responsive models (PMID: 28803728, 31636208).

Interestingly, the ISG+ Tregs found in B16F10 decreased after monotherapy and were completely depleted after combination therapy, which is consistent with previous studies (PMID: 16778987, 23897981, 24777248). On the other hand, the ST2+ Tregs present in mT4 tumors remained in the TME after both single agent and combination ICT treatments. The findings suggest that the presence of distinct Treg cells with specific phenotypes in the TME of ICT-responsive and non-responsive tumors may indicate a potential resistance mechanism to ICT response.

### Example 5

### Longitudinal Tumor Analyses Highlight Progressive Increase of Suppressive CD4 and CD8 T Cells and Intratumoral Tregs with Enhanced Suppressive Phenotypes in Mice Bearing Pancreatic Tumors

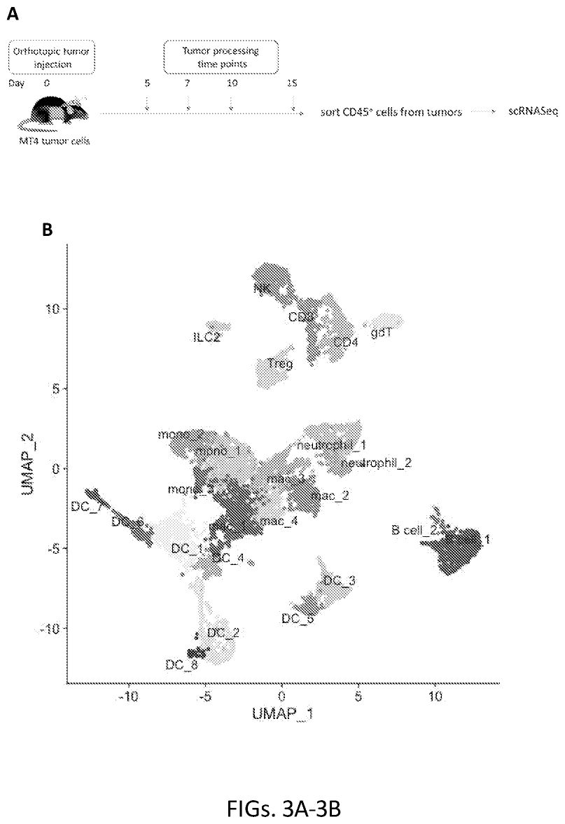

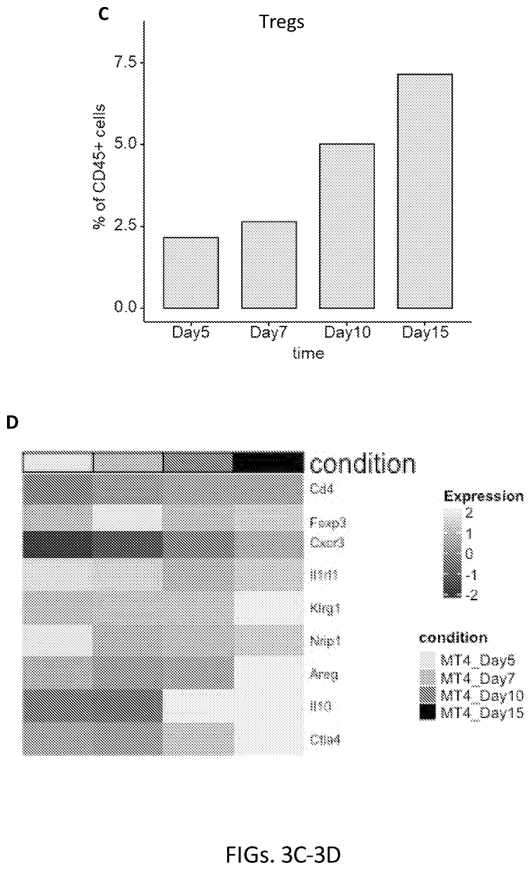

To determine when ST2+Tregs infiltrated pancreatic tumors and showed enhanced suppressive capacity, the inventors performed a longitudinal assessment of the immune microenvironment at different time points from Day 5 to Day 15 of tumor growth (FIG. 3A-B). The inventors observed an exponential increase in Tregs from early to late-stage tumors (FIG. 3C), and gene expression analysis revealed that the Tregs expressed ST2 (gene il1rl1) throughout the progression analysis (FIG. 3D). Additionally, Klrg1, Areg1, Il10, and Ctla4expression increased dramatically in late-stage tumors (FIG. 3D). The data demonstrate that T cells in the pancreatic TME have pro-tumor phenotypes and that ST2+ Tregs are present from early-stage tumors and increase in both abundance and suppressive capacity as tumors progress.

### Example 6

### Analysis of Patient Samples at Baseline Highlights Presence of ST2+Tregs in Non-Responders

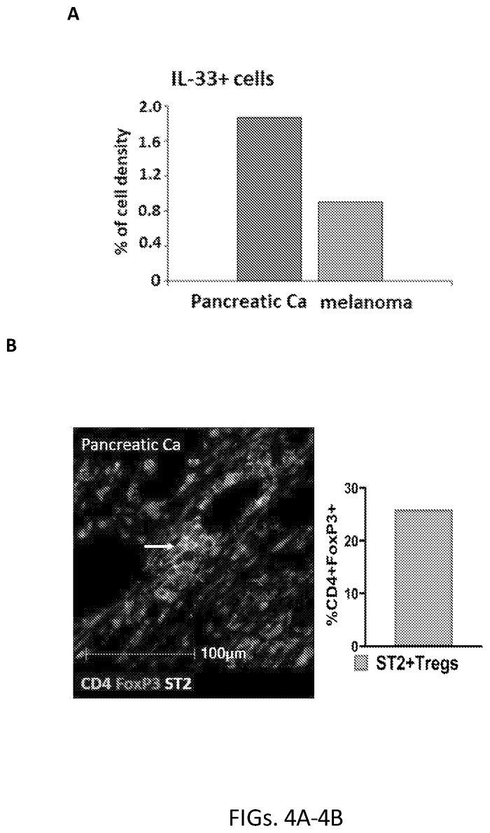

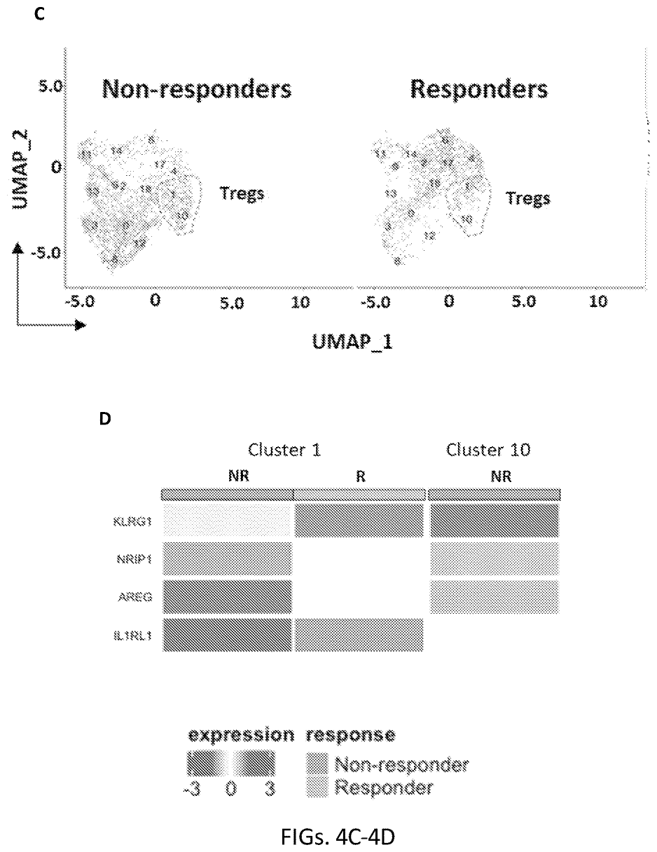

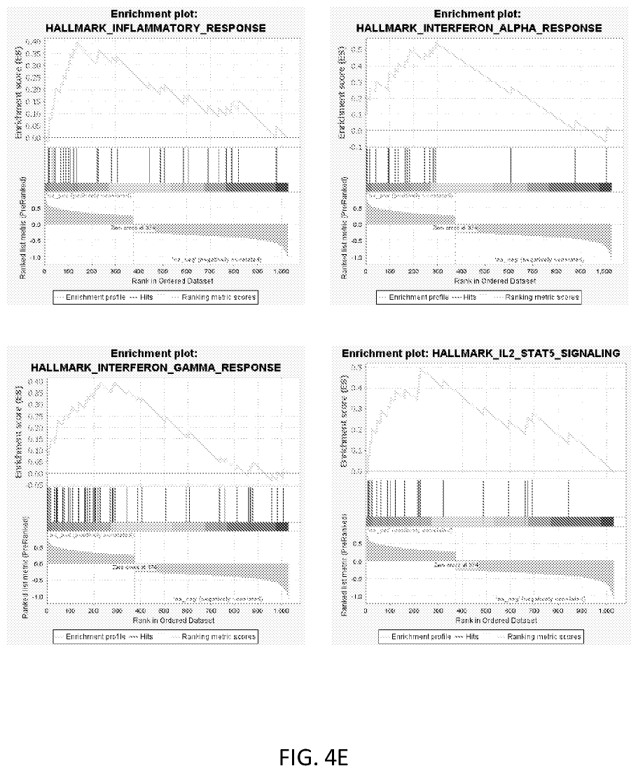

To investigate whether the presence of ST2+ Tregs could indicate response to ICT, the inventors first assessed IL33 levels, the chemokine that recruits ST2+ Tregs in pancreatic and melanoma patient tissues. Elevated IL-33 was identified in pancreatic tissues and multi-immunofluorescence performed on the pancreatic tissues indicated the presence of ST2+ Tregs in the pancreatic tumors (FIG. 4A-B). Next, to determine if the ICT response is independent of the tumor type but only dictated by the presence of ST2+ Tregs, the inventors re-analyzed a publicly available dataset of responders (R) and non-responders (NR) of patients with advanced melanoma at baseline (PMID: 30388456) to determine if the ICT response is independent of the tumor type but only dictated by the presence of ST2+Tregs (FIG. 4C). Two distinct subsets of Tregs (Cluster 1 and 10) were identified, with only Cluster 1 present in baseline responders. Tregs expressing IL1RL1, NRP1, KLRG1, AREG1 genes observed in the mT4 Tregs were specifically present in the Tregs in the non-responders (FIG. 4D). GSEA analysis showed higher interferon gamma, interferon alpha, and IL2-STAT5 signaling in Cluster 1, indicative of Treg subsets similar to those seen in B16F10 tumors (FIG. 4E). Therefore, the presence of ST2+ Tregs at baseline in patient samples can potentially determine the responsiveness of patients to ICT.

### Example 7

### Discussion of Examples Herein

Our data demonstrate that the T cell populations in tumors responsive or resistant to ICT are fundamentally different at baseline, and this is the key determinant of response to ICT. Previous studies have suggested that the presence of specific CD8 T cell phenotypes is a crucial factor in determining the efficacy of ICT response (PMID: 30388456, among others). In ICT-sensitive B16F10 melanoma tumors, the CD8 T cell subsets are more effector and progenitor-like, possessing characteristics that promote anti-tumor function, consistent with the changes observed following ICT and improved survival outcomes. In contrast, mT4 tumors have fewer CD8 T cells and more suppressive Th2 cells at baseline. In contrast to B16F10 tumors, CD8 T cell levels did not increase after ICT treatment, consistent with the lack of ICT efficacy and poor survival outcomes observed in these mice.

Despite increasing knowledge about CD8 and CD4 T cell heterogeneity, the heterogeneity of Tregs is poorly understood. Recent studies have shown that Tregs exhibit tissue-specific gene signatures (PMID: 30737144), but tumor-specific Treg heterogeneity and its effect on ICT response have not been extensively studied. The inventors identified two distinct subsets of Tregs in ICT-sensitive and resistant tumors. The former was dominated by ISG+Tregs that were depleted after ICT, while the latter consisted of ST2+Tregs that expressed klrg1, areg, and other suppressive genes and did not deplete post-ICT therapy. ST2, also known as IL-33R, interacts with IL-33 in the TME and promotes pro-tumor functions in Tregs (PMID: 31165767, 32878747, 32246094). However, the role of ST2 in the context of ICT has not been well studied. A recent study in a murine model of lung adenocarcinoma demonstrated the presence of klrg1+areg+il1rl1+ Tregs, similar to the phenotype observed in mT4 tumors, in advanced tumors and associated with poor cancer prognosis (PMID: 31801068). Furthermore, deletion of Treg-specific ST2 altered Treg phenotype and reduced tumor burden and CD8 T cell infiltration. The longitudinal analysis of pancreatic tumors showed that ST2+Tregs were present from early stages of tumor initiation and increased exponentially as tumors progressed. This suggests that targeting these subsets early in tumor progression may be important to increase CD8 T cell infiltration and ICT efficacy. The inventors observed the presence of ST2+Tregs in baseline melanoma patients who did not respond to ICT. Thus, ST2 can be used as a marker to identify patients who will benefit from ICT plus agents targeting Tregs.

In this study, the inventors conducted a comprehensive analysis of intratumoral T cell subsets and identified distinct ST2+Treg subsets in ICT-nonresponsive tumors. These findings emphasize the importance of Treg heterogeneity in determining ICT response and highlight the potential utility of Treg phenotypes as a biomarker for predicting ICT response.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Son J, Cho JW, Park HJ, et al. Tumor-Infiltrating Regulatory T-cell Accumulation in the Tumor Microenvironment Is Mediated by IL33/ST2 Signaling. Cancer Immunol Res. 2020;8(11):1393-1406. doi:10.1158/2326-6066.CIR-19-0828
2. Li A, Herbst RH, Canner D, et al. IL-33 Signaling Alters Regulatory T Cell Diversity in Support of Tumor Development. Cell Rep. 2019;29(10):2998-3008.e8. doi:10.1016/j.celrep.2019.10.120
3. Cui, G., Yuan, A., Li, Z. et al. ST2 and regulatory T cells in the colorectal adenoma/carcinoma microenvironment: implications for diseases progression and prognosis. Sci Rep 10, 5892 (2020). https://doi.org/10.1038/s41598-020-62502-0
4. Van der Jeught K, Sun Y, Fang Y, et al. ST2 as checkpoint target for colorectal cancer immunotherapy. JCI Insight. 2020;5(9):e136073. Published 2020 May 7. doi:10.1172/jci.insight.136073

What is claimed is:

1. A method of detecting ST2 in a population of Tregs, the method comprising the step of detecting ST2 in a biological sample comprising Tregs from an individual, wherein the individual has a cancer that is a candidate for an immune checkpoint therapy.

2. The method of claim 1, wherein the biological sample comprising Tregs cells comprises a tumor biopsy.

3. The method of claim 1 or 2, wherein the biological sample is enriched for lymphocytes.

4. The method of any one of claims 1-3, wherein the biological sample is enriched for T-cells.

5. The method of any one of claims 1-4, wherein the biological sample is enriched for Tregs.

6. The method of claim 1 or 2, wherein the biological sample is prepared for immunohistochemistry.

7. The method of any one of claims 1-6, wherein detecting ST2 comprises detecting by immunohistochemistry.

8. The method of any one of claims 1-6, wherein detecting ST2 comprises detecting by sequencing.

9. The method of claim 8, wherein the sequencing comprises RNA sequencing.

10. The method of claim 9, wherein the RNA sequencing comprises single cell RNA sequencing.

11. The method of any one of claims 1-10, wherein the immune checkpoint therapy comprises an anti-PD1 composition and/or an anti-CTLA4 composition.

12. A method of treating a cancer in an individual, the method comprising the step of administering a non-immune checkpoint therapy to an individual who has been determined to have ST2+ Tregs in an amount substantially equal to or greater than a reference level.

13. The method of claim 12, wherein the reference level is a detectable amount of ST2+ Tregs present in the individual.

14. The method of claim 12, wherein the reference level is determined by measuring ST2+ Tregs in two or more biological samples from the individual.

15. The method of claim 12, wherein the reference level comprises an amount of ST2+ Tregs found in an individual responsive to an immune checkpoint therapy or a healthy individual.

16. A method of treating a cancer in an individual, the method comprising the step of administering an immune checkpoint therapy to an individual who has been determined to have ST2+ Tregs in an amount less than a reference level.

17. The method of claim 16, wherein the reference level is a detectable amount of ST2+ Tregs present in the individual.

18. The method of claim 16, wherein the reference level is determined by measuring ST2+ Tregs in two or more biological samples from the individual.

19. The method of claim 16, wherein the reference level comprises an amount of ST2+ Tregs found in an individual responsive to an immune checkpoint therapy or a healthy individual.

20. A method of prognosing the effectiveness of an immune checkpoint therapy in an individual, the method comprising the step of measuring the presence of ST2+ Tregs to get a measurement and comparing the measurement to a reference.

21. The method of claim 20, wherein the immune checkpoint therapy comprises an anti-PD1 composition and/or an anti-CTLA4 composition.

22. The method of claim 20 or 21, wherein the reference is a detectable amount of ST2+ Tregs present in the individual.

23. The method of claim 22, wherein the reference is determined from measuring ST2+ Tregs in two or more biological samples from the individual.

24. The method of claim 22, wherein the reference comprises an amount of ST2+ Tregs found in an individual responsive to an immune checkpoint therapy or a healthy individual.

25. A method of treating a cancer in an individual, the method comprising the step of administering a composition comprising a therapeutically effective amount of an anti-ST2 agent.

26. The method of claim 25, wherein the anti-ST2 composition comprises an antibody targeting ST2, an RNA interference composition targeting ST2, and/or a small molecule capable of inhibiting ST2.

27. The method of claim 25 or 26, further comprising administering a therapeutically effective amount of an immune checkpoint therapy.

28. The method of claim 27, wherein the composition comprises the immune checkpoint therapy.

29. The method of claim 27, wherein the composition does not comprise the immune checkpoint therapy.

30. The method of claim 27, wherein the composition and immune checkpoint therapy are given concurrently.

31. The method of claim 27, wherein the composition and immune checkpoint therapy are given sequentially.

32. The method of any one of claims 27-30, wherein the immune checkpoint therapy comprises an anti-PD1 composition and/or an anti-CTLA4 composition.

33. The method of any one of claims 25-32, wherein the cancer is refractory to and immune checkpoint therapy.

34. The method of any one of claims 25-33, wherein the cancer is a pancreatic cancer and/or melanoma

35. A method for determining a treatment for an individual with cancer, the method comprising the step of administering a non-checkpoint inhibitor therapy to an individual found to have an amount of ST2+ Tregs equal to or greater than a reference.

36. The method of claim 35, wherein the detectable amount of ST2+ Tregs is found by measuring the expression of ST2 in at least one biological sample from the individual.

37. The method of claim 36, wherein the biological sample comprises a tumor biopsy.

38. The method of claim 36 or 37, wherein the biological sample is enriched for lymphocytes.

39. The method of any one of claims 36-38, wherein the biological sample is enriched for T-cells.

40. The method of any one of claims 36-38, wherein the biological sample is enriched for Tregs.

41. The method of claim 36 or 37, wherein the biological sample is prepared for immunohistochemistry.

42. The method of any one of claims 35-41, wherein the reference is a detectable amount of ST2+ Tregs present in the individual.

43. The method of claim 42, wherein the reference is determined from measuring ST2+ Tregs in two or more biological samples from the individual.

44. The method of claim 42, wherein the reference comprises an amount of ST2+ Tregs found in an individual responsive to an immune checkpoint therapy or a healthy individual.
